(19) United States
(12) Patent Application Publication
(10) Pub. No.: US 2009/0012101 A1
(43) Pub. Date: Jan. 8, 2009

(54) Mucarinic Agonists and Methods of Use Thereof

(75) Inventors: William S. Messer, JR., Toledo, OH (US); Yang Cao, Scotch Plains, NJ (US); Frederick Tejada, Toledo, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: UNIVERSITY OF TOLEDO, Toledo, OH (US)

(21) Appl. No.: 12/159,426

(22) PCT Filed: Dec. 8, 2006
(86) PCT No.: PCT/US06/46840

§ 371 Date: Jun. 27, 2008

Related U.S. Application Data
(60) Provisional application No. 60/754,529, filed on Dec. 27, 2005.

(51) Int. Cl. A61K 31/439; C07D 453/02; A61K 31/506; A61P 25/18; C07D 401/14

(52) U.S. Cl. 514/256; 546/133; 546/112; 544/333; 514/305; 514/299

(57) ABSTRACT

A method of forming analogs of CDD-0304, i.e., tetra(ethyleneglycol) (4-methoxy-1,2,5-thiadiazol-3-yl)[3-(1-methyl-1,2,4,5-tetrahydropyrid-3-yl)-1,2,5-thiadiazol-4-yl]ether hydrochloride includes one or more of the following steps: a) replacing the tetrahydropyridine moiety with one of the following heterocyclic rings, including quinuclidine, [2.2.1]-exo-azabicy-cloheptane, [2.2.1]-endo-azabicycloheptane and terahydropyrimidine; b) varying the length of the linking group by replacing the tetra(ethylene) glycol moiety with one of: ethylene glycol, di(ethylene) glycol, penta(ethylene) glycol, or diether diol; and/or, c) replacing the 1,2,5-thiadiazole moiety with an ester isostere. Also, a method for an asymmetric analog CCD-0304 includes replacing at least one moiety with an ester isostere and at least a second moiety with an ammonium isostere. Also, such analogs compounds and their uses are disclosed.

### BACKGROUND OF THE INVENTION

Schizophrenia is a psychiatric disorder that afflicts approximately two million Americans. The yearly cost to society for patient care is estimated to be $23 billion per year, the United States alone. Approximately $2.3 billion was spent on antipsychotic medications in 1999, and the antipsychotic drug market is expected to grow to more than $6 billion by 2006.

The underlying cause of schizophrenia is unknown, although an imbalance of activity at dopamine synapses has been proposed to play a role in the positive symptoms such as hallucinations, delusions and disordered thought patterns.^{1,2} For many years, dopamine antagonists such as haloperidol and chlorpromazine have been widely used in the treatment of schizophrenia. Unfortunately, many of the classical antipsychotic compounds produce unwanted side effects such as Parkinsonism, acute dystonia and akathisia. The adverse effects are mainly due to blockade of dopamine (D₂) receptors, although many antipsychotic compounds interact with other G protein-coupled receptors. The classical antipsychotics are effective in treating the positive symptoms of schizophrenia, but are much less useful in treating negative symptoms such as social withdrawal and blunted affect and exacerbate the cognitive deficits associated with schizophrenia.

In the 1980s, the atypical antipsychotic clozapine was found to be effective in treating both positive and negative symptoms of schizophrenia with a relatively lower incidence of extrapyramidal side effects. Despite its effectiveness in treating schizophrenia patients, the clinical utility of clozapine is limited by the development of agranulocytosis in a small subset (0.6%) of patients. Other atypical antipsychotics, including olanzapine, risperidone and quetiapine, may be as effective as clozapine in treating the positive symptoms of schizophrenia, yet do not produce agranulocytosis. Over the past decade, therapeutic approaches toward the treatment of schizophrenia have focused on developing more effective antipsychotic compounds with reduced side effect profiles.

Although a number of treatments are available to treat the positive and negative symptoms, relatively few efforts have focused on developing compounds that can improve cognitive disturbances associated with schizophrenia.³ Cognitive deficits, including impaired working memory, attention and executive functions, are strongly linked to the long-term disabilities found in patients with schizophrenia.⁴ The cognitive deficits associated with schizophrenia represent an important clinical target for drug development that has not been addressed by the pharmaceutical industry.

The dopamine hypothesis of schizophrenia has had pronounced effect on the strategies for developing antipsychotics.¹⁸ However, even recent atypical antipsychotics do not completely relieve all schizophrenic symptoms.¹⁹

While recent advances have been made in the understanding of the cholinergic nervous system and the receptors therein, there is still a need to develop compositions which do not have the adverse affects of the currently available treatment regimes. One avenue being pursued involves cholinergic receptors which are proteins embedded in the cell membrane that respond to the chemical acetylcholine. Cholinergic receptors are subdivided into the nicotinic and muscarinic receptor families, and muscarinic receptors represent a family of five subtypes.

Muscarinic receptors mediate a variety of physiological responses to the neurotransmitter acetylcholine in the central and peripheral nervous systems. M₁ muscarinic receptors play a role in learning and memory function in the brain and regulate gastric acid secretion in the stomach. M₂ receptors regulate neurotransmitter release in the central nervous system and control cardiac muscle contraction. Acetylcholine stimulates smooth muscle contraction in a variety of tissues and promotes secretion from exocrine glands. These effects are mediated by M₃ receptors. Though less well characterized pharmacologically, M₄ receptors appear to play a role in the perception of pain, and M₅ receptors may regulate dopaminergic activity in the brain.

Consequently, muscarinic receptors are being considered as important targets in the treatment of schizophrenia. The M₄ muscarinic receptor plays an important role in balancing dopamine activity indicating a potential use of M₄ agonists in treating psychosis.²⁰ A role for M₂ receptors also has been proposed.²¹

While an M₁//M₂/M₄ agonist profile could provide efficacy in a broad range of symptomatic domains of schizophrenia including enhancement of cognitive function¹⁴, the development of selective muscarinic agonists has been hindered by the high degree of homology among the five receptor subtypes.

Anticholinergic drugs, including the glycolate esters, produce psychotomimetic effects in humans.⁵ Muscarinic antagonists have been used in schizophrenic patients to control the parkinsonism associated with administration of antipsychotics with dopamine antagonist activity, yet at higher doses, muscarinic antagonists exacerbate the symptoms of schizophrenia, producing confusion and hallucinations. In contrast, Alzheimer's disease patients treated with cholinesterase inhibitors, which elevate levels of acetylcholine, exhibit improvements in neuropsychiatric symptoms such as agitation, hallucinations and psychosis.⁶⁻⁸ In addition, the selective M₁/M₄ muscarinic agonist xanomeline significantly improved psychiatric symptoms such as hallucinations in phase II clinical trials in Alzheimer's patients.⁹ Unfortunately, xanomeline produced unwanted side effects associated with activation of M₃ receptors, including salivation, diarrhea and profuse sweating, that limited patient compliance.¹⁰ The side effects seem to be associated with rapid metabolism of the alkyloxy side chain following oral administration, resulting in a nonselective, yet active compound with limited therapeutic utility. Despite a second phase II clinical trial with a patch formulation, the liabilities of xanomeline still outweigh its benefits.

Follow-up preclinical studies with structurally related compounds identified strong antipsychotic activity in (5R,6R)-6-(3-butylthio-1,2,5-thiadiazol-4-yl)-1-azabicyclo[3.2.1]octane (BuTAC), which displays partial agonist activity at M₂ and M₄ receptors.¹¹ Muscarinic agonists such as xanomeline and BuTAC may exert an antipsychotic action by regulating the release of dopamine in the frontal cortex.¹²⁻¹⁴ Xanomeline and BuTAC produce very few of the adverse side effects (e.g., catalepsy) associated with classical antipsychotics such as haloperidol, suggesting that selective muscarinic agonists might provide a useful alternative therapeutic approach to treating the symptoms of schizophrenia. Moreover, muscarinic agonists might be particularly useful in improving cognitive function (including memory function, language use and constructional praxis) in schizophrenic patients.³

Behavioral studies of muscarinic receptor knockout mice also suggest the utility of M₁ and M₄ agonists in the treatment of psychosis.¹⁴ For example, M₄ receptors modulate locomotor activity produced by the stimulation of D₁ dopamine receptors. M₄ knockout mice also show enhanced sensitivity to the effects of PCP on the pre-pulse inhibition model of psychosis. Since M₁ and M₂ receptors play a role in cognitive and memory function,^{15,16} agonists with M₁ and M₂ activity might be particularly useful in treating memory and cognitive deficits associated with schizophrenia.^{3,14}

Several of the co-inventors herein have discovered muscarinic agonists, which are claimed in U.S. Pat. Nos. 6,096,767; 6,211,204 B1; 6,376,675 B2; 6,369,081 B1; and 6,602,891 B2, which are expressly incorporated herein by reference.

Efforts to develop selective muscarinic agonists have been hampered by the high degree of amino acid homology within the binding pocket of muscarinic receptors. Compounds with larger size and functional groups that interact with the extracellular loops of muscarinic receptors may interact with unique amino acid residues and selectively activate M₁ and M₄ receptors.

In view of the foregoing, it would be desirable to provide muscarinic agonists that result in the selective activation of muscarinic receptors, particularly so side effects are minimized during treatment of the conditions noted above.

Thus, there is a need for muscarinic agonists with activity at M₁, M₂ and M₄ receptors which then would useful in the treatment of Alzheimer's disease and schizophrenia, and other cognitive impairment disorders.

It is another object of the present invention to provide compounds that activate M₁, M₂ and M₄ receptors, which enhance memory function and modulate dopamine function, respectively.

It is another object of the present invention to provide compounds having improved muscarinic receptor selectivity profiles (specifically at M₁, M₂ and M₄ receptors).

It is another object of the present invention to provide pharmaceutical composition comprising compounds of the present invention, as active ingredients.

### SUMMARY OF THE INVENTION

In another aspect, the present invention relates to analogs of the potent muscarinic receptor agonist, CDD 0304, tetra(ethylene glycol)(4-methoxy-1,2,5-thiadiazol-3-yl)[3-methyl-1,2,5,6-tetrahyrdopyrid-3-yl)-1,2,5,thiadiazol-4-yl]ether hydrochloride.

In another aspect, the present invention relates to a method of forming analogs of CDD-0304, i.e., tetra(ethyleneglycol) (4-methoxy-1,2,5-thiadiazol-3-yl)[3-(1-methyl-1,2,4,5-tetrahydropyrid-3-yl)-1,2,5-thiadiazol-4-yl]ether hydrochloride comprising at least one of the following steps:
a) replacing the tetrahydropyridine moiety with one of the following heterocyclic rings, including quinuclidine, [2.2.1]-exo-azabicycloheptane, [2.2.1]-endo-azabicycloheptane and terahydropyrimidine;
b) varying the length of the linking group by replacing the tetra(ethylene) glycol moiety with one of: ethylene glycol, di(ethylene) glycol, penta(ethylene) glycol, or diether diol; and/or,
c) replacing the 1,2,5-thiadiazole moiety with an ester isostere.

In another aspect, the present invention relates to a method for an asymmetric analog of tetra(ethyleneglycol) (4-methoxy-1,2,5-thiadiazol-3-yl)[3-(1-methyl-1,2,4,5-tetrahydropyrid-3-yl)-1,2,5-thiadiazol-4-yl]ether hydrochloride comprising: replacing in formula Scheme 5, the A moiety with an ester isostere and B moiety with various ammonium isostere.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 is an illustration of Scheme 1, showing the synthesis of tetra(ethyleneglycol) (4-methoxy-1,2,5-thiadiazol-3-yl)[3-(1-methyl-1,2,4,5-tetrahydropyrid-3-yl)-1,2,5-thiadiazol-4-yl]ether hydrochloride (CCD-0304).

FIG. 2 is an illustration of Scheme 2 showing various heterocyclic rings which replace the tetrahydropyridine moiety found in CCD-0304.

FIG. 3 is an illustration of Scheme 3 showing a diether diol useful as a linking group.

FIG. 4 is an illustration of Scheme 4 showing the various esters which replace the 1,2,5-thiadiazole moiety in CCD-0304.

FIG. 5 is an illustration of Scheme 5 showing an asymmetric analog of a muscarinic agonist.

FIG. 6A shows: Tables 1a—Effect of spacer length on the inhibition of [³H]-(R)-QNB binding to human muscarinic receptor subtypes expressed in A9 cells. FIG. 6B shows—Table 1b. Effect of methoxy positioning and tripropylene glycol spacer on inhibition of [³H]-(R)-QNB binding to human muscarinic receptor subtypes expressed in A9 cells; and, FIG. 6C shows—Table 1 c. Effect of basic terminal group on the inhibition of [³H]-(R)-QNB binding to human muscarinic receptor subtypes expressed in A9 cells.

FIG. 7 shows: Table 2—Stimulation of PI metabolism through activation of M₁, M₃ and M₅ receptors and inhibition of adenylyl cyclase activity through activation of M₂ and M₄.

FIG. 8 shows: Table 3—Stimulation of PI metabolism through activation of M₁, M₃ and M₅ receptors and inhibition of adenylyl cyclase activity through activation of M₂ and M₄.

### DETAILED DESCRIPTION OF THE PREFERRED EMBODIMENT

In one aspect, the present invention relates to compounds which have an improved M₁/M₄ agonist activity and selectivity and CNS penetration. The compounds are designed and synthesized based on structural modifications of the compound CDD-0304.

CDD-0304 consists of the agonist pharmacophore, i.e., xanomeline, which is linked via an ethylene glycol spacer to a terminal 3-methoxy-1,2,5-thiadiazole ring. Xanomeline can undergo N-oxidation and N-demethylation at the tetrahydropyridine ring which affects its muscarinic agonist properties. According to one aspect of the present invention, in order to prevent this N-oxidation and N-demethylation, the N-methyl group is incorporated in a ring, thus giving various azabicyclic and tricyclic systems.

In one aspect, the present invention relates to a method for forming a first set of compounds which have a suitable ammonium bioisostere in place of the tetrahydropyridine ring in order to improve agonist activity and selectivity, and to the compounds formed thereby.

In another aspect, the present invention relates to a method for forming a second set of compounds which have different linking groups, and the compounds formed thereby. The linking groups are both varied in the nature and length of the linking group.

In yet another aspect, the present invention relates to a method for forming analogs of CCD-0304 where the terminal thiadiazole moiety of CDD-0304 is replaced with various heterocyclic rings. The analogs improve M₁, M₂ and/or M₄ agonist activity and selectivity and CNS penetration.

In still another aspect, the present invention relates to CDD-0304 analogs which optimize M₁, M₂ and M₄ agonist activity and selectivity and CNS penetration for the treatment of various neurological and psychiatric disorders such as Alzheimer's disease and schizophrenia.

In one aspect, disclosed herein is a method of increasing the activity of a muscarinic receptor comprising contacting the receptor with an effective amount of at least one CCD-0304 analog compound.

In another aspect, disclosed herein is a method of treating a subject suffering from a muscarinic receptor related disorder comprising identifying a subject in need thereof and administering to the subject a therapeutically effective amount of at least one CCD-0304 analog compound. By “muscarinic related disorder,” it is meant a disorder whose symptoms are ameliorated by activating a muscarinic receptor.

In another aspect, disclosed herein is a method of treating schizophrenia or psychosis of any origin in a subject, comprising identifying a subject in need thereof and administering to the subject a therapeutically effective amount of at least one CDD-0304 analog compound. In some embodiments, the method comprises treating a subject with a pharmacologically active dose of at least one CDD-0304 analog compound, for the purpose of controlling the positive (hallucinations and delusion), negative (apathy, social withdrawal, anhedonia) and cognitive symptoms of schizophrenia or related psychosis.

In another aspect, the present invention relates to a method of ameliorating at least one symptom in a subject of a condition where it is beneficial to increase the level of activity of at least one of an M₁, M₂ and/or M₄ muscarinic receptor comprising: determining that the subject would benefit from an increased level of activity of at least one of an M₁ M₂ and/or M₄ muscarinic receptor; and administering an amount of at least one analog of the 1,2,5,6-tetrahydropyridine compound CDD-0304 which is therapeutically effective to increase the level of activity of said at least one of an M₁, M₂ and/or M₄ muscarinic receptor and to ameliorate said at least one symptom to the subject.

In certain embodiments, it is within the scope of the contemplated invention that the CDD-0304 analog compounds may be administered in a single daily dose, or the total daily dosage may be administered as a plurality of doses, (e.g., divided doses two, three or four times daily). Furthermore, compounds for the present invention may be administered in intranasal form via topical use of suitable intranasal vehicles, or via transdermal routes, or via topical use of ocular formulations, or using those forms of transdermal skin patches well known to persons skilled in the art.

It is to be further understood that the dosage regimen can be selected in accordance with a variety of factors. These include type, species, age, weight, sex and medical condition of the patient; the severity of the condition to be treated; the route of administration; the kidney and liver functions of the patient; and the particular compounds employed. A physician of ordinary skill can readily determine and prescribe the effective amount of the drug required to prevent, counter or arrest the progress of the disease or disorder that is being treated. For example, the daily dosage of the products may be varied over a wide range from 0.01 to 1000 mg per adult human per day. An effective amount of the drug is ordinarily supplied at a dosage level of about 0.0001 mg/kg to about 25 mg/kg body weight per day. Preferably, the range is from about 0.001 to 10 mg/kg of body weight per day, and especially from about 0.001 mg/kg to 1 mg/kg of body weight per day. The compounds may be administered on a regimen of 1 to 4 times per day.

It is also within the contemplated scope of the present invention that the CDD-0304 analog compounds may be used alone at appropriate dosages defined by routine testing in order to obtain optimal pharmacological effect, while minimizing any potential toxic or otherwise unwanted effects. In addition, the CDD-0304 analog compounds may be used as adjunctive therapy with known drugs to reduce the dosage required of these traditional drugs, and thereby reduce their side effects.

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated.

The term “pharmaceutically acceptable addition salts” refers to salts known in the art to be acceptable in pharmaceutical practice, for example acid addition salts such as hydrochloric acid salts, maleic acid salts, and citric acid salts. Pharmaceutically acceptable acid addition salts include salts derived form inorganic acids such as hydrochloric, nitric, phosphoric, sulfuric, hydrobromic, hydroiodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are the salts of amino acids such as arginate, gluconate, galacturonate, and the like.

The term “metabolite” refers to a form of a compound obtained in a human or animal body by action of the body on the administered form of the compound, for example a de-methylated analogue of a compound bearing a methyl group which is obtained in the body after administration of the methylated compound as a result of action by the body on the methylated compound. Metabolites may themselves have biological activity.

The term “prodrug” refers to a form a compound which after administration to a human or animal body is converted chemically or biochemically to a different compound in said body having biological activity. A prodrug form of a compound may itself have biological activity.

The novel compounds of embodiments of the present invention, and compounds which may be used in accordance with embodiments of the present invention may have at least one chiral center, and may accordingly exist as enantiomers or as mixtures of enantiomers (e.g., racemic mixtures). Where the compounds possess two or more chiral centers, they may additionally exist as diastereoisomers.

In some embodiments of the present invention, there are provided pharmaceutical compositions and the use of certain compounds in the manufacture of pharmaceutical compositions. Such compositions may be in a form suitable for oral (e.g., in the form of capsules, tablets, granules, powders or beads), rectal, parenteral, intravenous, intradermal, subcutaneous, transdermal or topical administration, or for administration by insufflation or nasal spray, iontophoretic, buccal, or sublingual lingual administration. Such compositions may be in unit dosage form. Certain of the compounds in some embodiments of the present invention can exist in unsolvated forms as well as solvated forms, including hydrated forms. In general, the solvated forms, including hydrated forms, are equivalent to unsolvated forms and are intended to be encompassed within the scope of the present invention.

### EXAMPLES

The following is a detailed example of a preferred process to prepare compounds described herein. It will be understood that the following examples are not intended to limit the scope of the invention.

The compounds described herein can be prepared as described in the schemes shown in the figures. For example, the 1,2,5,6-tetrahydropyridine compounds were prepared by the Williamson ether formation method to incorporate the thiadiazoles with the corresponding n-(ethylene glycol) linkers, where n is the number of ethylene glycol unit. The intermediate 3-(3-chloro-1,2,5-thiadiazol-4-yl)pyridine was synthesized using the published procedure.²³ The intermediate 5-chloro-3-methoxy-1,2,4-thiadiazole was prepared using previous procedure.²⁴

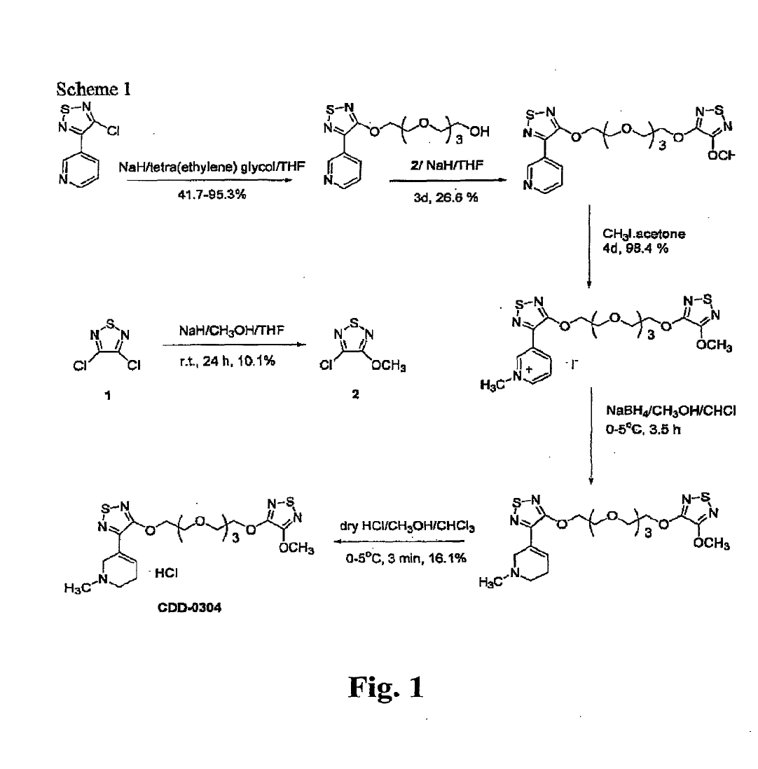

FIG. 1 is an illustration of Scheme 1, showing the synthesis of 1,2,5,6-tetrahydropyridine compound CCD-0304 having an ethylene glycol spacer as shown, where n=3, R is 1 or 2. General Procedure: (i) NaH/THF, ethylene glycol, reflux (ii) NaH/THF, thiadiazole moiety (iii) CH₃I, acetone, rt (iv) NaBH₄/MeOH.CHCl₃, 0-5° C. (v) HCl.

### Example 1

### Synthesis of CDD-0304

The general scheme for the synthesis of CDD-0304 is shown in FIG. 1. The methods were adapted from those reported previously for bivalent muscarinic agonists such as CDD-0273.

### Example 2

### Ammonium Isosteres

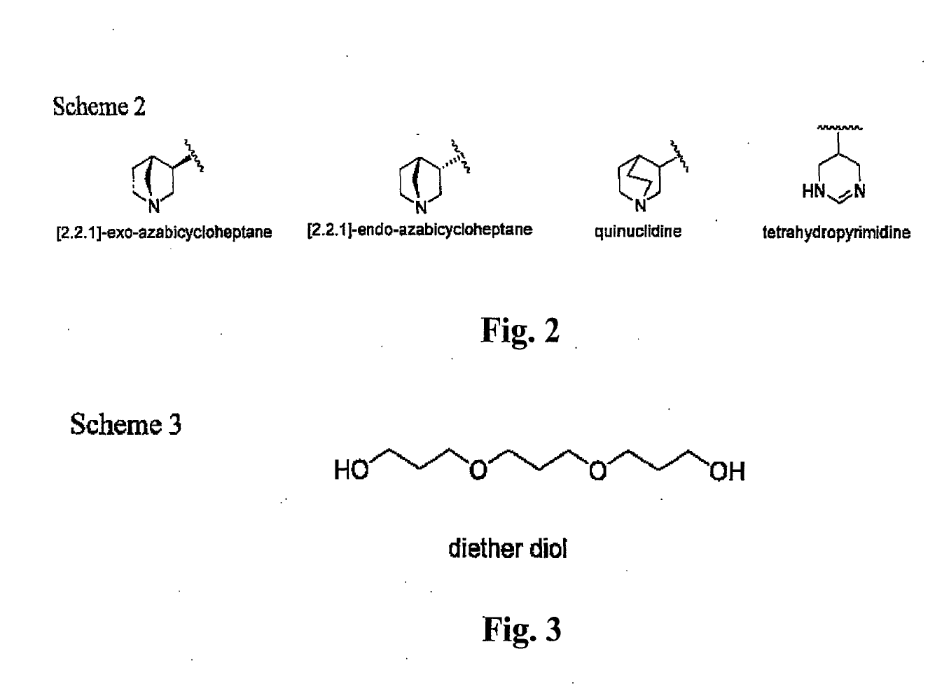

The tetrahydropyridine found in CDD-0304 is replaced with one of the following heterocyclic rings, including quinuclidine, [2.2.]-exo-azabicycloheptane, [2.2.1]-endo-azabicycloheptane and terahydropyrimidine, as shown in FIG. 2. The appropriate 4-butyl-sulfonyl-1,2,5-thiadiazolyl substituted heterocyclic rings are readily synthesized according to established methods, and used as starting materials for subsequent incorporation of the ethylene glycol linking group and the ester isostere.

### Example 3

### Linking Group

The lengths of the linking group with ethylene glycol derivative is varied. For example, ethylene glycol, di(ethylene) glycol or penta(ethylene) glycol is incorporated in place of tetra(ethylene) glycol. Diether diol, an analogous linking group to tetra(ethylene) glycol having 13 atoms but with a relatively rigid property, is also useful ad a linking group used, as shown in FIG. 3.

### Example 4

### Ester Isosteres

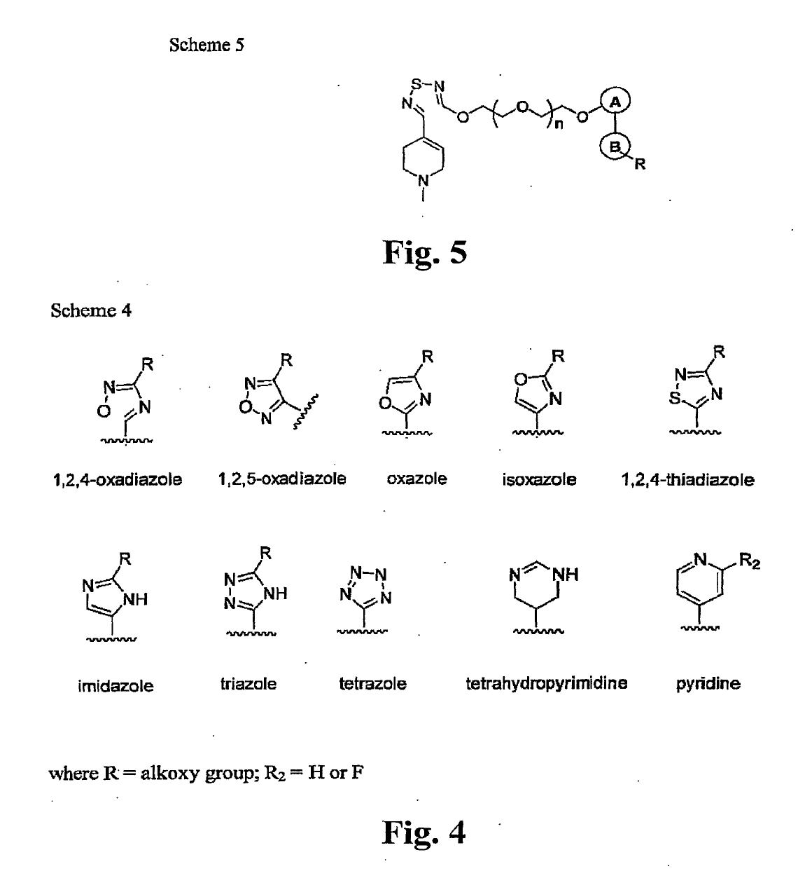

A wide variety of ester isosteres are utilized including 5 and 6 carbons cycloalkyl with one or more heteroatoms selected from N, S or 0 as replacements for the 1,2,5-thiadiazole moiety found in CDD-0304, as shown in FIG. 4. Examples of the ester isosteres include: 1,2,4-oxodiazole, 1,2,5-oxadiazole, oxazole, isoazole, 1,2,4-thiadiazole, imidizole, triazole, tetrazole, tetrahydropyrimidine, and pyridine. The hydrogen bond interactions are optimized with amino acid residues on the M₁ and M₄ receptors, thereby improving selectivity.

### Example 5

### Asymmetric Analogs

To further exploit the muscarinic agonist properties of the bivalent ligand approach, asymmetric analogs are synthesized, as shown in FIG. 5, where A is the one of various ester isosteres and B is the one of various ammonium isosteres both described above. [R═CH₃ for tetrahydropyrimidine or R═F for pyridine].

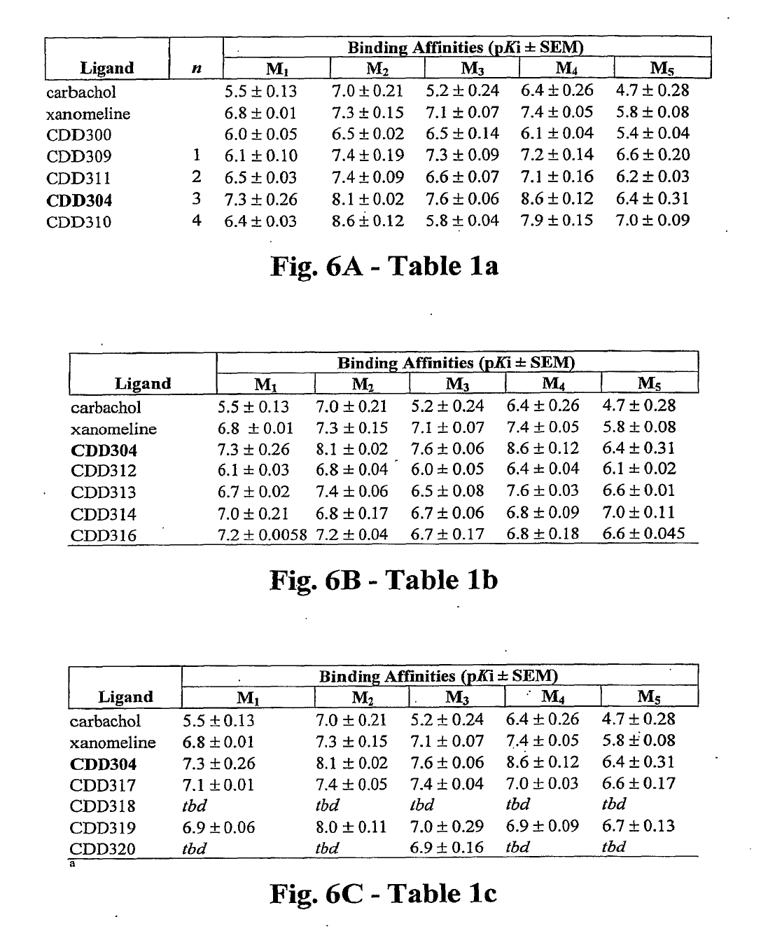

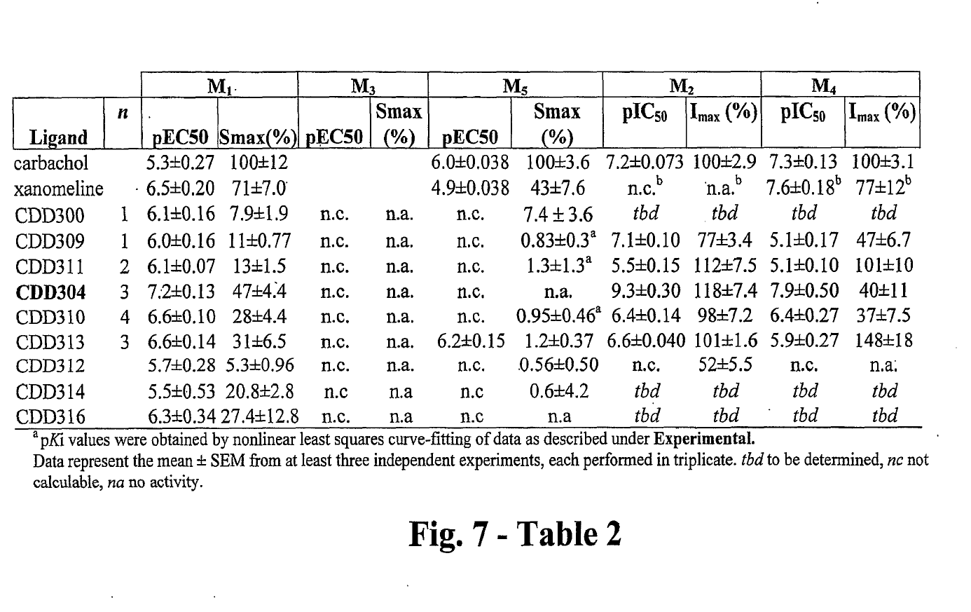

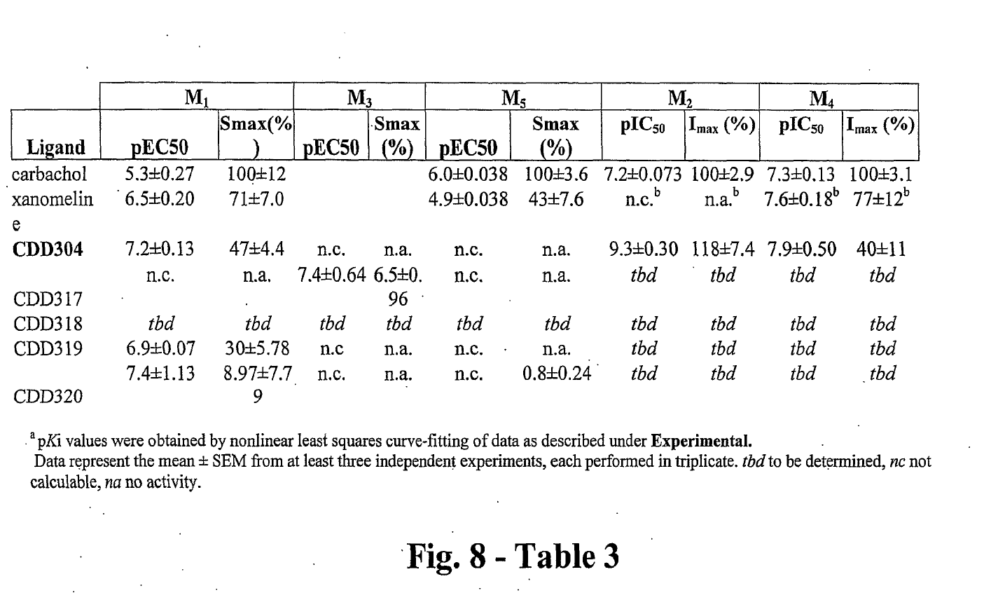

The ligand binding affinities (pKᵢ values) of the synthesized compounds were determined and are shown in Tables 1a to 1c in FIGS. 6A-6C. The functional properties of the novel compounds at M₁, M₃ and M₅ muscarinic receptors were determined using muscarinic receptor-mediated PI hydrolysis assays. For the M₂ and M₄ receptors, ligands were investigated for their ability to inhibit forskolin-stimulated cAMP accumulation. Carbachol was utilized in each assay as a positive control for muscarinic receptor activation (as shown in Tables 2 and 3 in FIGS. 7 and 8, respectively).

The skilled artisan will appreciate that many factors influence the selection of any compound for application in clinical therapy, e.g., effectiveness for the intended purpose, safety, possible side-effects and therapeutic index. The skilled artisan will thus appreciate how to interpret the expression “pharmaceutically acceptable quaternary compounds” which are structurally derived from the inventive compounds having a tertiary nitrogen atom, as this expression is used in the present specification and claims.

While the invention as been described with reference to a preferred embodiment, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed as the best mode contemplated for carrying out this invention, but that the invention will include all embodiments falling within the scope of the claims.

Each of the following references is incorporated by reference herein in its entirety, including any drawings.
(1) Carlsson, A.; Waters, N.; Holm-Waters, S.; Tedroff, J.; Nilsson, M. et al. Interactions between monoamines, glutamate, and GABA in schizophrenia: new evidence. Annu Rev Pharmacol Toxicol 2001, 41, 237-260.
(2) Carlsson, A. The current status of the dopamine hypothesis of schizophrenia. Neuropsychopharmacology 1988, 1, 179-186.
(3) Friedman, J. I.; Temporini, H.; Davis, K. L. Pharmacologic strategies for augmenting cognitive performance in schizophrenia. Biol Psychiatry 1999, 45, 1-16.
(4) Hyman, S. E.; Fenton, W. S. Medicine. What are the right targets for psychopharmacology? Science 2003, 299, 350-351.
(5) Abood, L. G.; Biel, J. H. Anticholinergic psychotomimetic agents. Int Rev Neurobiol 1962, 4, 217-273.
(6) Cummings, J. L.; Back, C. The cholinergic hypothesis of neuropsychiatric symptoms in Alzheimer's disease. Am J Geriatr Psychiatry 1998, 6, S64-78.
(7) Levy, M. L.; Cummings, J. L.; Kahn-Rose, R. Neuropsychiatric symptoms and cholinergic therapy for Alzheimer's disease. Gerontology 1999, 45 Suppl 1, 15-22.
(8) Cummings, J. L. The role of cholinergic agents in the management of behavioural disturbances in Alzheimer's disease. Int J Neuropsychopharmacol 2000, 3, 21-29.
(9) Bodick, N. C.; Offen, W. W.; Shannon, H. E.; Satterwhite, J.; Lucas, R. et al. The selective muscarinic agonist xanomeline improves both the cognitive deficits and behavioral symptoms of Alzheimer disease. Alzheimer Dis Assoc Disord 1997, 11, S16-22.
(10) Bodick, N. C.; Offen, W. W.; Levey, A. I.; Cutler, N. R.; Gauthier, S. G. et al. Effects of xanomeline, a selective muscarinic receptor agonist, on cognitive function and behavioral symptoms in Alzheimer disease. Arch Neurol 1997, 54, 465-473.
(11) Rasmussen, T.; Fink-Jensen, A.; Sauerberg, P.; Swedberg, M. D.; Thomsen, C. et al. The muscarinic receptor agonist BuTAC, a novel potential antipsychotic, does not impair learning and memory in mouse passive avoidance. Schizophr Res 2001, 49, 193-201.
(12) Jones, C. K.; Shannon, H. E. Effects of scopolamine in comparison with apomorphine and phencyclidine on prepulse inhibition in rats. Eur J Pharmacol 2000, 391, 105-112.
(13) Jones, C. K.; Shannon, H. E. Muscarinic cholinergic modulation of prepulse inhibition of the acoustic startle reflex. J Pharmacol Exp Ther 2000, 294, 1017-1023.
(14) Felder, C. C.; Porter, A. C.; Skillman, T. L.; Zhang, L.; Bymaster, F. P. et al. Elucidating the role of muscarinic receptors in psychosis. Life Sci 2001, 68, 2605-2613.
(15) Anagnostaras, S. G.; Murphy, G. G.; Hamilton, S. E.; Mitchell, S. L.; Rahnama, N. P. et al. Selective cognitive dysfunction in acetylcholine M1 muscarinic receptor mutant mice. Nat Neurosci 2003, 6, 51-58.
(16) Seeger, T.; Fedorova, I.; Zheng, F.; Miyakawa, T.; Koustova, E. et al. M2 muscarinic acetylcholine receptor knock-out mice show deficits in behavioral flexibility, working memory, and hippocampal plasticity. J Neurosci 2004, 24, 10117-10127.

1. A method for forming an analog of tetra(ethyleneglycol) (4-methoxy-1,2,5-thiadiazol-3-yl)[3-(1-methyl-1,2,4,5-tetrahydropyrid-3-yl)-1,2,5-thiadiazol-4-yl]ether hydrochloride comprising: replacing the tetrahydropyridine moiety with one of the following heterocyclic rings, including quinuclidine, [2.2.1]-exo-azabicycloheptane, [2.2.1]-endo-azabicycloheptane and terahydropyrimidine.

2. A method for forming an analog of tetra(ethyleneglycol) (4-methoxy-1,2,5-thiadiazol-3-yl)[3-(1-methyl-1,2,4,5-tetrahydropyrid-3-yl)-1,2,5-thiadiazol-4-yl]ether hydrochloride comprising: varying the length of the linking group by replacing the tetra(ethylene) glycol moiety with one of: ethylene glycol, di(ethylene) glycol, penta(ethylene) glycol, or diether diol.

3. A method for an analog of tetra(ethyleneglycol) (4-methoxy-1,2,5-thiadiazol-3-yl)[3-(1-methyl-1,2,4,5-tetrahydropyrid-3-yl)-1,2,5-thiadiazol-4-yl]ether hydrochloride comprising: replacing the 1,2,5-thiadiazole moiety with an ester isostere.

4. The method of claim 3, wherein in the ester isostere comprises a 5 or 6 carbon cycloalkyl having one or more heteroatoms selected from N, S or 0.

5. The method of claim 4, wherein the ester isostere includes: 1,2,4-oxodiazole, 1,2,5-oxadiazole, oxazole, isoazole, 1,2,4-thiadiazole, imidizole, triazole, tetrazole, tetrahydropyrimidine, or pyridine.

6. A method for an asymmetric analog of tetra(ethyleneglycol) (4-methoxy-1,2,5-thiadiazol-3-yl)[3-(1-methyl-1,2,4,5-tetrahydropyrid-3-yl)-1,2,5-thiadiazol-4-yl]ether hydrochloride comprising: replacing in formula Scheme 5, the A moiety with an ester isostere and B moiety with various ammonium isostere.

7. An analog of tetra(ethyleneglycol) (4-methoxy-1,2,5-thiadiazol-3-yl) [3-(1-methyl-1,2,4,5-tetrahydropyrid-3-yl)-1,2,5-thiadiazol-4-yl]ether hydrochloride wherein the tetrahydropyridine moiety is replaced with one of the following heterocyclic rings, including quinuclidine, [2.2.1]-exo-azabicycloheptane, [2.2.1]-endo-azabicycloheptane and terahydropyrimidine.

8. An analog of tetra(ethyleneglycol) (4-methoxy-1,2,5-thiadiazol-3-yl)[3-(1-methyl-1,2,4,5-tetrahydropyrid-3-yl)-1,2,5-thiadiazol-4-yl]ether hydrochloride wherein the tetra(ethylene) glycol moiety is replaced with one of: ethylene glycol, di(ethylene) glycol, penta(ethylene) glycol, or diether diol.

9. An analog of tetra(ethyleneglycol) (4-methoxy-1,2,5-thiadiazol-3-yl) [3-(1-methyl-1,2,4,5-tetrahydropyrid-3-yl)-1,2,5-thiadiazol-4-yl]ether hydrochloride wherein the 1,2,5-thiadiazole moiety is replaced with an ester isostere.

10. The analog of claim 9, wherein the ester isostere comprises a 5 or 6 carbon cycloalkyl having one or more heteroatoms selected from N, S or 0.

11. The analog of claim 9, wherein the ester isostere includes: 1,2,4-oxodiazole, 1,2,5-oxadiazole, oxazole, isoazole, 1,2,4-thiadiazole, imidizole, triazole, tetrazole, tetrahydropyrimidine, or pyridine.

12. An asymmetric analog of tetra(ethyleneglycol) (4-methoxy-1,2,5-thiadiazol-3-yl)[3-(1-methyl-1,2,4,5-tetrahydropyrid-3-yl)-1,2,5-thiadiazol-4-yl]ether hydrochloride wherein the compound of Scheme 5 has the A moiety replaced with an ester isostere and B moiety with various ammonium isostere.

13. A method of activating at least one of an M₁, M₂ and/or M₄ muscarinic receptor in a subject comprising contacting said receptor with at least one analog of claim 7.

14. A method of ameliorating at least one symptom in a subject of a condition where it is beneficial to increase the level of activity of at least one of an M1 and/or M4 muscarinic receptor comprising: determining that the subject would benefit from an increased level of activity of at least one of an M₁, M₂ and/or M₄ muscarinic receptor; and administering an amount of at least one at least one analog of claim 7, which is therapeutically effective to increase the level of activity of said at least one of an M₁, M₂ and/or M₄ muscarinic receptor and to ameliorate said at least one symptom to the subject.

15. The method of claim 14, wherein the subject suffers from a condition selected from the group consisting of hallucinations, delusions, disordered thought, behavioral disturbance, aggression, suicidality, mania, impaired cognitive function, schizophrenia, and two or more any of the foregoing conditions.

16. A pharmaceutical composition for treating schizophrenia comprising an efficacious amount of at least one analog of claim 7 and at least one pharmaceutically acceptable carrier, diluent or excipient therefore.

17. A method of treating schizophrenia, comprising administering to a patient in need thereof an efficacious amount of an analog of claim 7 and pharmaceutically acceptable salts thereof and mixtures or salts thereof.

18. A method of ameliorating symptoms of schizophrenia, comprising administering to a patient in need thereof an efficacious amount of an analog of claim 7 and pharmaceutically acceptable salts thereof and mixtures or pharmaceutically acceptable salts thereof.

19. The method of claim 17 wherein the subject is human.

20. The method of claim 17, further comprising contacting said subject with an additional therapeutic agent.

21. A compound of claim 7, or an acid addition salt, solvate or hydrate thereof.

22. A compound of claim 8, or an acid addition salt, solvate or hydrate thereof.

23. A compound of claim 9, or an acid addition salt, solvate or hydrate thereof.

24. A compound of claim 10, or an acid addition salt, solvate or hydrate thereof.

25. A compound of claim 11, or an acid addition salt, solvate or hydrate thereof.

26. A compound of claim 12, or an acid addition salt, solvate or hydrate thereof.

27. A pharmaceutical composition, comprising a therapeutically effective amount of a compound according to claim 7, and a pharmaceutically acceptable carrier.

28. A pharmaceutical composition, comprising a therapeutically effective amount of a compound according to claim 8, and a pharmaceutically acceptable carrier.

29. A pharmaceutical composition, comprising a therapeutically effective amount of a compound according to claim 9, and a pharmaceutically acceptable carrier.

30. A pharmaceutical composition, comprising a therapeutically effective amount of a compound according to claim 10, and a pharmaceutically acceptable carrier.

31. A pharmaceutical composition, comprising a therapeutically effective amount of a compound according to claim 11, and a pharmaceutically acceptable carrier.

32. A pharmaceutical composition, comprising a therapeutically effective amount of a compound according to claim 12, and a pharmaceutically acceptable carrier.

33. A method of activating at least one of an M₁, M₂ and/or M₄ muscarinic receptor in a subject comprising contacting said receptor with at least one analog of claim 8.

34. A method of ameliorating at least one symptom in a subject of a condition where it is beneficial to increase the level of activity of at least one of an M1 and/or M4 muscarinic receptor comprising: determining that the subject would benefit from an increased level of activity of at least one of an M₁, M₂ and/or M₄ muscarinic receptor; and administering an amount of at least one at least one analog of claim 8 which is therapeutically effective to increase the level of activity of said at least one of an M₁, M₂ and/or M₄ muscarinic receptor and to ameliorate said at least one symptom to the subject.

35. A pharmaceutical composition for treating schizophrenia comprising an efficacious amount of at least one analog of claim 8 and at least one pharmaceutically acceptable carrier, diluent or excipient therefore.

36. A method of treating schizophrenia, comprising administering to a patient in need thereof an efficacious amount of an analog of claim 8 and pharmaceutically acceptable salts thereof and mixtures or salts thereof.

37. A method of ameliorating symptoms of schizophrenia, comprising administering to a patient in need thereof an efficacious amount of an analog of claim 8 and pharmaceutically acceptable salts thereof and mixtures or pharmaceutically acceptable salts thereof.

38. A method of activating at least one of an M₁, M₂ and/or M₄ muscarinic receptor in a subject comprising contacting said receptor with at least one analog of claim 9.

39. A method of ameliorating at least one symptom in a subject of a condition where it is beneficial to increase the level of activity of at least one of an M1 and/or M4 muscarinic receptor comprising: determining that the subject would benefit from an increased level of activity of at least one of an M₁, M₂ and/or M₄ muscarinic receptor; and administering an amount of at least one at least one analog of claim 9 which is therapeutically effective to increase the level of activity of said at least one of an M₁, M₂ and/or M₄ muscarinic receptor and to ameliorate said at least one symptom to the subject.

40. A pharmaceutical composition for treating schizophrenia comprising an efficacious amount of at least one analog of claim 9 and at least one pharmaceutically acceptable carrier, diluent or excipient therefore.

41. A method of treating schizophrenia, comprising administering to a patient in need thereof an efficacious amount of an analog of claim 9 and pharmaceutically acceptable salts thereof and mixtures or salts thereof.

42. A method of ameliorating symptoms of schizophrenia, comprising administering to a patient in need thereof an efficacious amount of an analog of claim 9 and pharmaceutically acceptable salts thereof and mixtures or pharmaceutically acceptable salts thereof.

43. A method of activating at least one of an M₁, M₂ and/or M₄ muscarinic receptor in a subject comprising contacting said receptor with at least one analog of claim 10.

44. A method of ameliorating at least one symptom in a subject of a condition where it is beneficial to increase the level of activity of at least one of an M1 and/or M4 muscarinic receptor comprising: determining that the subject would benefit from an increased level of activity of at least one of an M₁, M₂ and/or M₄ muscarinic receptor; and administering an amount of at least one at least one analog of claim 10 which is therapeutically effective to increase the level of activity of said at least one of an M₁, M₂ and/or M₄ muscarinic receptor and to ameliorate said at least one symptom to the subject.

45. A pharmaceutical composition for treating schizophrenia comprising an efficacious amount of at least one analog of claim 10 and at least one pharmaceutically acceptable carrier, diluent or excipient therefore.

46. A method of treating schizophrenia, comprising administering to a patient in need thereof an efficacious amount of an analog of claim 10 and pharmaceutically acceptable salts thereof and mixtures or salts thereof.

47. A method of ameliorating symptoms of schizophrenia, comprising administering to a patient in need thereof an efficacious amount of an analog of claim 10 and pharmaceutically acceptable salts thereof and mixtures or pharmaceutically acceptable salts thereof.

48. A method of activating at least one of an M₁, M₂ and/or M₄ muscarinic receptor in a subject comprising contacting said receptor with at least one analog of claim 11.

49. A method of ameliorating at least one symptom in a subject of a condition where it is beneficial to increase the level of activity of at least one of an M1 and/or M4 muscarinic receptor comprising: determining that the subject would benefit from an increased level of activity of at least one of an M₁, M₂ and/or M₄ muscarinic receptor; and administering an amount of at least one at least one analog of claim 11 which is therapeutically effective to increase the level of activity of said at least one of an M₁, M₂ and/or M₄ muscarinic receptor and to ameliorate said at least one symptom to the subject.

50. A pharmaceutical composition for treating schizophrenia comprising an efficacious amount of at least one analog of claim 11 and at least one pharmaceutically acceptable carrier, diluent or excipient therefore.

51. A method of treating schizophrenia, comprising administering to a patient in need thereof an efficacious amount of an analog of claim 11 and pharmaceutically acceptable salts thereof and mixtures or salts thereof.

52. A method of ameliorating symptoms of schizophrenia, comprising administering to a patient in need thereof an efficacious amount of an analog of claim 11 and pharmaceutically acceptable salts thereof and mixtures or pharmaceutically acceptable salts thereof.

53. A method of activating at least one of an M₁, M₂ and/or M₄ muscarinic receptor in a subject comprising contacting said receptor with at least one analog of claim 12.

54. A method of ameliorating at least one symptom in a subject of a condition where it is beneficial to increase the level of activity of at least one of an M1 and/or M4 muscarinic receptor comprising: determining that the subject would benefit from an increased level of activity of at least one of an M₁, M₂ and/or M₄ muscarinic receptor; and administering an amount of at least one at least one analog of claim 12 which is therapeutically effective to increase the level of activity of said at least one of an M₁, M₂ and/or M₄ muscarinic receptor and to ameliorate said at least one symptom to the subject.

55. A pharmaceutical composition for treating schizophrenia comprising an efficacious amount of at least one analog of claim 12 and at least one pharmaceutically acceptable carrier, diluent or excipient therefore.

56. A method of treating schizophrenia, comprising administering to a patient in need thereof an efficacious amount of an analog of claim 12 and pharmaceutically acceptable salts thereof and mixtures or salts thereof.

57. A method of ameliorating symptoms of schizophrenia, comprising administering to a patient in need thereof an efficacious amount of an analog of claim 12 and pharmaceutically acceptable salts thereof and mixtures or pharmaceutically acceptable salts thereof.

58. The method of claim 18, wherein the subject is human.

59. The method of claim 18, further comprising contacting said subject with an additional therapeutic agent.
